(19) United States
(12) Patent Application Publication
(10) Pub. No.: US 2007/0178149 A1
(43) Pub. Date: Aug. 2, 2007

(54) Levodopa compositions

(76) Inventors: Moshe Flashner-Barak, Petach Tikva (IL); E. Itzhak Lerner, Petach Tikva (IL); Vered Rosenberger, Modiin (IL)

Correspondence Address: KENYON & KENYON LLP, ONE BROADWAY, NEW YORK, NY, 10004, US

(21) Appl. No.: 11/594,307

(22) Filed: Nov. 7, 2006

Related U.S. Application Data
(60) Provisional application No. 60/734,684, filed on Nov. 7, 2005.

(51) Int. Cl. A61K 31/195; A61K 9/14; A61K 9/24; A61K 9/54

(52) U.S. Cl. 424/458; 424/472; 424/484; 514/567

(57) ABSTRACT

The invention provides compositions of levodopa resulting in extended absorption profiles and methods of treatment using the compositions.

### RELATED APPLICATIONS

This application claims the benefit of U.S. provisional Application Ser. No. 60/734,684, filed Nov. 7, 2005, hereby incorporated by reference.

### FIELD OF THE INVENTION

The invention encompasses compositions that provide a delayed and/or extended absorption of levodopa.

### BACKGROUND OF THE INVENTION

Parkinson's disease is a degenerative condition associated with reduced dopamine concentrations in the basal ganglia region of the brain. The deficiency is thought to be caused by oxidative degradation of dopaminergic neurons in the substantia nigra. One course of therapy is restoring the dopamine concentration in the brain by administrating levodopa, a metabolic precursor of dopamine that, unlike dopamine, is able to cross the blood-brain barrier. Levels of dopamine in the brain reportedly follow the blood levels of levodopa, because levodopa in the blood is the main source of dopamine in the brain for patients suffering from Parkinson's disease.

The metabolic transformation of levodopa to dopamine is catalyzed by the aromatic L-amino acid decarboxylase enzyme. This enzyme is found throughout the body including gastric juices and the mucosa of the intestine. The possibility of extracerebral metabolism of levodopa necessitates administration of large doses of the drug. As high extracerebral concentrations of dopamine cause nausea in some patients, levodopa is usually administered with an inhibitor of the aromatic L-amino decarboxylase enzyme, such as carbidopa. Organic acids have been used to enhance the stability of carbidopa in formulations of levodopa and carbidopa, as disclosed in U.S. Pat. No. 6,531,153.

Administration of levodopa is a treatment rather than a cure for Parkinson's disease. There are also certain difficulties associated with treatment of Parkinson's disease using levodopa. For example, patients typically experience a cycling between “on” and “off” states as the blood plasma concentration of levodopa rises and falls during treatment. In the “on” state, disease symptoms are suppressed by levodopa; however, when the blood plasma concentration of levodopa drops, the patient enters the so called “off state,” and symptoms of the disease return. Additionally, there is a delay between the time of ingesting levodopa and a return to the “on state,” upon administering a dose of levodopa. Prolonged treatment of Parkinson's disease with levodopa typically also results in the brain becoming less sensitive to levodopa, necessitating more frequent dosing with the drug to suppress the manifestations of the disease, which include tremor, muscular rigidity, lack of facial expression, and altered gait. However, aggressive administration of levodopa to circumvent off state symptoms can lead to equally disabling involuntary motions called dyskinesias.

Another problem in Parkinson's disease therapy is the reduction in plasma levodopa concentration that occurs while a patient is sleeping. Parkinson's patients usually awaken in the morning in the off state and must wait for a morning dose of levodopa to take effect before they can function comfortably.

From the foregoing, it will be appreciated that it is highly desirable to be able to administer levodopa as a sustained release oral dosage form capable of stabilizing the serum level of levodopa in a patient. It would be highly desirable if a Parkinson's disease patient could take levodopa in the evening, while under the therapeutic effect of a previous dose, and wake up in the morning without the manifestations of the disease. For such purpose, the drug delivery vehicle ideally would not only extend the release of levodopa over time, but would also delay release of levodopa until the early morning hours before the patient awakens so that the patient would awaken when the therapeutic effect of the dose is near its maximum.

Such controlled release formulations have been used in an attempt to maintain relatively constant the blood concentration of levodopa in patients. U.S. Pat. Nos. 4,832,957 and 4,900,755 describe controlled release formulations for the delivery of a combination of levodopa and carbidopa. A formulation of levodopa with carbidopa is currently available in Sinemet® CR controlled release tablets (DuPont Pharma). According to the Physician's Desk Reference, 54th ed., the tablets use a polymeric based drug delivery system that allows the tablets to slowly erode, releasing the actives. U.S. Pat. Nos. 6,238,699 and 6,756,056 disclose formulations containing an immediate release layer of levodopa and carbidopa combined with a controlled release layer of the two drugs. The immediate release portion is apparently efficiently absorbed into the body but the controlled release portion is not.

Moreover, the presumed mechanism of absorption of levodopa from the gastrointestinal tract limits the effectiveness of such controlled release formulations in providing prolonged suppression of disease manifestations. Levodopa is absorbed in the stomach and by the active transport mechanism for amino acids, which is most active in the duodenum region of the small intestine. However, the residence time of a dosage through the duodenum is very short; typically, the time a drug remains in the duodenum is measured in minutes rather than hours. It is believed that the efficiency of the transport of the amino acids of this sort in the jejunum and ileum is considerably lower than in the duodenum. Sustained release is therefore limited by the transit time of the dosage through the stomach and duodenum which, though highly variable from individual-to-individual and dependent upon nutritional state, typically takes only about 3 to 4 hours. Levodopa released after the 3-4 hour therapeutic window has passed is believed to be essentially not bioavailable. For example, Sinemet® CR controlled release tablets have about 75% of the bioavailability of Sinemet® conventional release tablets. Physicians Desk Reference, 54th edition (Medical Economics Co., publisher, 2000) at p. 979.

One possible method of extending the time of drug absorption in the body is by using a gastric retention system of the dosage form to retain the dosage form in the gastrointestinal tract for a longer period of time, and thereby increase the time for absorption. U.S. publication No. 2004/0234608, published Nov. 25, 2004, discloses a composition that expands rapidly upon contacting gastric fluid which may be used in formulations of levodopa to retain the drug in the stomach. The composition contains a hydrogel network and tannic acid, which is used to aid in the rapid expansion of the hydrogel network. This composition increases in volume about three fold within about 15 minutes of contacting gastric fluid, thus keeping the levodopa in the stomach for a longer time of absorption in the duodenum.

Another approach to achieving improved results with levodopa is inhibiting the elimination of levodopa from the body, thus, increasing the lifetime of the drug in the body and its subsequent bioavailability. This approach consists of the incorporation of another drug besides carbidopa that works to inhibit a levodopa elimination mechanism. One such drug is entacapone which apparently inhibits the COMT, catechol-O-methyltransferase, mechanism of levodopa metabolism. Drug combinations of levodopa, carbidopa, and entacapone have been described in U.S. Pat. Nos. 6,500,867 and 6,797,732 and are marketed under the name Stalevo.® In this treatment method, tablets of levodopa and carbidopa can apparently be dosed separately from the entacapone tablet. While this drug combination extends the time of levodopa in the blood by slowing its elimination, the dosing of another active material creates the potential for added side effects and may cause the inhibition of another enzyme system whose proper working is necessary for normal health.

We have found that levodopa which is purposely released after the duodenum, is absorbed into the bloodstream more efficiently than previously thought. The appearance of this levodopa in the blood is delayed compared to the levodopa found in the blood after the dosing of immediate release or controlled release levodopa.

### SUMMARY OF THE INVENTION

One aspect of the invention encompasses compositions and dosage forms comprising levodopa in an amount of about 30 mg to about 500 mg wherein at least about 30 mg to about 500 mg of the levodopa is in first a reservoir that substantially does not release levodopa until after leaving the stomach and passing the duodenum, and about 0 mg to about 470 mg of the levodopa is in a second reservoir that releases levodopa in the stomach and/or the duodenum. The levodopa in the first reservoir for releasing after the duodenum may be in an immediate release formulation or a controlled release formulation. The release after the duodenum may be accomplished by using enteric coatings and/or other delay release coatings. The levodopa in the second reservoir for drug release in the stomach and/or the duodenum may be in an immediate release formulation or a controlled release formulation. The composition may further comprise at least one carboxylase enzyme inhibitor. Optionally, the composition further comprises a COMT inhibitor such as entacapone.

Another aspect of the invention comprises methods of treating Parkinson's disease comprising administering compositions and/or dosage forms comprising levodopa in an amount of about 30 mg to about 500 mg, wherein at least about 30 mg to about 500 mg of the levodopa is in a first reservoir that does not release drug until after leaving the stomach and passing the duodenum, and about 0 mg to about 470 mg of the levodopa is in a second reservoir that releases levodopa in the stomach and/or the duodenum. The levodopa in the first reservoir, which is released after the duodenum, may be released as an immediate release formulation or a controlled release formulation. So too, the levodopa in the second reservoir, which is released in the stomach and/or the duodenum, may be released as an immediate release or controlled released formulation. The composition may further comprise at least one carboxylase enzyme inhibitor and/or a COMT inhibitor.

### BRIEF DESCRIPTION OF THE FIGURES

FIG. 1 illustrates the plotted Averaged Data of a Test Formulation vs. the data of the Reference formulation as obtained in Example 4.

### DESCRIPTION OF THE PREFERRED EMBODIMENTS

Drugs that absorb throughout the length of the small intestine, but not in the colon, can be designed to deliver a zero order slow release profile over about five hours, i.e. the time of transit through the small intestine (three to four hours) plus the time the drug resides in the stomach, if drug release starts in the stomach. For drugs absorbed in the small intestine and colon, zero order release profiles of 12 hours and more are desired and reportedly usually achievable. However, a slow release form of levodopa that releases after the sum total of residence time in the stomach and duodenum is believed to waste the amount of drug released after passage through the duodenum.

The term “dose” is used herein to generally describe a portion of a drug to be delivered. The term “dosage form” is generally used to describe the delivery system used to deliver a dose of a drug, such as, but not limited to, a composition containing a dose of the drug comprised in a tablet.

As used herein, the term “immediate release” means that at least 85% of the drug is released in 60 minutes or less once drug release is initiated for the immediate release dose.

A levodopa metabolic precursor like the levodopa ethyl ester of U.S. Pat. No. 5,840,756, hereby incorporated by reference, may be substituted for levodopa in the various embodiments of the invention.

One aspect of this invention encompasses compositions and dosage forms comprising levodopa in an amount of about 30 mg to about 500 mg, wherein at least about 30 mg to about 500 mg of the levodopa is present in a first reservoir that substantially does not release levodopa until after leaving the stomach and passing the duodenum, and about 0 mg to about 470 mg of the dose is in a second reservoir that releases levodopa in the stomach and/or the duodenum. In one embodiment, the first reservoir, which substantially does not release levodopa until after passing the duodenum, releases the drug in the upper jejunum or about 10 to 40 minutes after passing from the stomach. In another embodiment, in the first reservoir the levodopa is released in the mid or end of the jejunum or about 40 to 80 minutes after passing from the stomach. In yet another embodiment, in the first reservoir the levodopa is released in the ileum or in the terminal ileum or about 80 to 240 minutes after passing from the stomach. In a more preferred embodiment, the levodopa is released in the upper or mid jejunum or about 10 to 60 minutes after passing from the stomach, and most preferably about 20 minutes after passing from the stomach. The first reservoir may deliver levodopa over a period of about 2 to about 5 hours.

Another embodiment of the invention includes an optional third reservoir of levodopa designed to release drug after leaving the small intestine and entering the colon. In one embodiment of the invention, the drug in the third reservoir may be an immediate release formulation or a controlled release formulation, and preferably the drug in the third reservoir is a controlled release formulation.

In one embodiment, the second drug reservoir for releasing levodopa in the stomach and/or the duodenum is an immediate release formulation or controlled release formulation. In a more preferred embodiment, the second reservoir for releasing levodopa in the stomach and/or the duodenum is a controlled release formulation. The second reservoir may deliver levodopa over a period of about 2 to about 5 hours.

Optionally, the composition may further comprise at least one decarboxylase enzyme inhibitor. Examples of decarboxylase enzyme inhibitors suitable for use in the composition include, but are not limited to, carbidopa or benserazide. The decarboxylase enzyme inhibitor may comprise about 3% to about 30% by weight of the composition, and preferably comprises about 10% to about 20% by weight of the composition. The decarboxylase enzyme inhibitor is typically present in an amount of about 10 mg to about 100 mg per dose, and preferably, present in an amount of about 50 mg. More preferably, the decarboxylase enzyme inhibitor is carbidopa and is present in an amount of about 50 mg per dose. In the preferred embodiments of the invention, decarboxylase enzyme inhibitors, such as carbidopa or benserazide, are incorporated in the same dosage form as the levodopa. Optionally, the composition may further comprise a COMT inhibitor such as entacapone. The COMT inhibitor may be present in an amount of about 50 mg to about 300 mg per dose, and most preferably about 200 mg.

One embodiment of the invention encompasses enteric coated compositions comprising levodopa in an amount of about 30 mg to about 500 mg, wherein the enteric coating substantially delays release of levodopa until after passage through the duodenum of a patient. Typically, the enteric coating is thick enough to delay release of levodopa. As used herein, the term “substantially delay release after passage through the duodenum” is defined as a composition having a drug release delay of at least about 10 minutes after passing the stomach; preferably, about 15 minutes to 60 minutes; and more preferably, about 20 minutes after passing from the stomach but less than 120 minutes after passing from the stomach. Such a delay may be demonstrated by an in vitro drug release delay of at least 10 minutes, or more preferably about 15 to 30 minutes after transferring the dosage form from a dissolution bath containing an acidic buffer to one containing a neutral buffer, such as phosphate buffer at pH 6.8. The dissolution bath followed the method described in USP Method II Paddles (900 mL at 37° C. and 50 rpm). See, USP §§<711> Dissolution and <724> Drug Release, pp. 2303 and 2305 (USP 27, 2004). Enteric coatings are well known in the pharmaceutical art and are exemplified by Eudragit L™, Eudragit S™, and Cellulose Acetate Phthalate.

In one embodiment, the compositions may be in the form of enteric coated tablets, enteric coated capsules, or enteric coated pellets. Tablets used in the invention may be bilayer tablets wherein the first and second reservoir are each a layer of the tablet. Most preferably, the compositions are in the form of enteric coated tablets. The composition may be an immediate release formulation or controlled release formulation and preferably it is a controlled release formulation. The enterically coated embodiments may further comprise at least one carboxylase enzyme inhibitor, wherein the carboxylase enzyme inhibitor is carbidopa or benserazide. Preferably, the carboxylase enzyme inhibitor is present in an amount of about 10 mg to about 100 mg per dose. Most preferably, the carboxylase enzyme inhibitor is carbidopa and is present in an amount of about 50 mg per dose. Optionally, the composition may further comprise a COMT inhibitor such as entacapone. The COMT inhibitor may be present in an amount of about 50 mg to about 300 mg per dose, and most preferably about 200 mg.

The enteric coated form of levodopa may have a second reservoir of levodopa comprising about 0 mg to 470 mg of levodopa formulated for releasing in the stomach and or duodenum. The second reservoir may be in the form of a film coating, powder coating, pressed coating, coating of pellets, separate layer or layers, embedded pellets, or packaged either together or separately as tablets or as pellets in a capsule. The drug in the second reservoir for releasing levodopa in the stomach and/or duodenum may be formulated as an immediate release composition or a controlled release composition. The second reservoir composition may further comprise at least one carboxylase enzyme inhibitor, wherein the carboxylase enzyme inhibitor carbidopa or benserazide. Preferably, the carboxylase enzyme inhibitor is present in an amount of about 10 mg to about 100 mg per dose. Most preferably, the carboxylase enzyme inhibitor is carbidopa and is present in an amount of about 50 mg per dose. Optionally, the composition may further comprise a COMT inhibitor such as entacapone. The COMT inhibitor may be present in amounts of about 50 mg to about 300 mg per dose, and most preferably about 200 mg.

In a preferred embodiment of this aspect of the invention, the first reservoir is an enteric coated tablet comprising about 30 to 500 mg of levodopa, and preferably, about 50 to 100 mg of levodopa. In one embodiment, the enteric coating is preferably thick enough to allow for an about 15 to 30 minute delay of drug release after leaving the stomach. In this preferred embodiment, the second reservoir is in the form of an outer annular sheath as described in U.S. application Ser. Nos. 10/419,536 entitled “Dosage Form with a Core Tablet of Active Ingredient Sheathed in a Compressed Angular Body of Powder or Granular Material, and Process and Tooling for Producing It,” filed on Apr. 21, 2003; Ser. No. 10/379,338 entitled “Controlled Release Dosage Forms,” filed on Mar. 3, 2003; and Ser. No. 11/190,766, entitled “Dosage Form with an Enterically Coated Core Tablet,” filed on Jul. 26, 2005, hereby incorporated by reference. The outer annular ring is formulated with levodopa and carboxylase enzyme inhibitor and formulated for either immediate release or sustained release delivery for the desired time. Most preferably, the outer ring is formulated as a short duration controlled release formulation. Typically, the outer sheath comprises about 0 mg to 470 mg of levodopa, and more preferably about 100 mg to 150 mg of levodopa. More preferably, the outer sheath further comprises about 50 mg of carbidopa. In one most preferable embodiment, the inner enteric coated tablet comprises 50 mg levodopa formulated to start levodopa release at least about 15 minutes after leaving the stomach and having a controlled release drug profile that releases levodopa over about three hours while the outer annular sheath comprises 150 mg levodopa and 50 mg carbidopa having a controlled release drug profile that starts drug release immediately upon entering the stomach and releases drug over about three hours.

In another most preferable embodiment the inner enteric coated tablet comprises about 100 mg of levodopa having a controlled release drug profile that starts drug release at least about 15 minutes after leaving the stomach and releases drug over about three hours and the outer annular sheath comprises 100 mg levodopa and 50 mg carbidopa having a controlled release drug profile that starts drug release immediately upon entering the stomach and releases drug over about three hours.

Another embodiment of this aspect of the invention encompasses delay coated compositions based on erosion of the coating. The embodiment comprises levodopa in an amount of about 30 mg to about 500 mg, wherein the delay coating substantially delays release of levodopa until after passage through the duodenum of a patient. In one embodiment, the enteric coating is thick enough to substantially delay release until after passage through the duodenum of the patient. The preferred times of drug release are the same as in the enteric coated embodiment. These compositions may be in the form of delay coated tablets, delay coated capsules, or delay coated pellets. Most preferably, the composition will be in the form of a delay coated tablet. Tablets used in the invention may be bilayer tablets wherein the first and second reservoir are each a layer of the tablet. The drug composition may be an immediate release formulation or a controlled release formulation and may further comprise at least one carboxylase enzyme inhibitor, wherein the carboxylase enzyme inhibitor is carbidopa or benserazide. The carboxylase enzyme inhibitor may be present in an amount of about 10 mg to about 100 mg per dose. Optionally, the composition may further comprise a COMT inhibitor such as entacapone. The COMT inhibitor may be present in amounts of about 50 mg to about 300 mg per dose, and most preferably, about 200 mg.

This delay coated form of levodopa may have a second reservoir of levodopa comprising 0 mg to 470 mg of levodopa formulated for releasing in the stomach and or duodenum. The delay coat may comprise a second reservoir of levodopa for release in the stomach and duodenum or may be separate therefrom. The second reservoir may be in the form of a film coating, powder coating, pressed coating, coating of pellets, separate layer or separate layers, embedded pellets, or packaged either together or separately as tablets or pellets in a capsule. The second reservoir drug composition for releasing levodopa in the stomach and/or duodenum may be an immediate release formulation or a controlled release formulation. Optionally, the second reservoir may further comprise at least one carboxylase enzyme inhibitor, wherein the carboxylase enzyme inhibitor is carbidopa or benserazide. The carboxylase enzyme inhibitor may be present in an amount of about 10 mg to about 100 mg per dose. Most preferably, the carboxylase enzyme inhibitor is carbidopa and is present in an amount of about 50 mg per dose. Optionally, the composition may further comprise a COMT inhibitor such as entacapone. The COMT inhibitor may be present in amounts of about 50 mg to about 300 mg per dose, and most preferably about 200 mg.

In a preferred embodiment of this aspect of the invention, the first reservoir of levodopa is a delay coated tablet comprising about 30 mg to 500 mg of levodopa, and preferably about 50 mg to 100 mg of levodopa. The delay coating substantially delays release of levodopa to after passage through the duodenum of a patient. Preferably, the delay coating is thick enough to allow for an about 15 to 30 minute delay of levodopa release after leaving the stomach.

Examples of delay coatings include, but are not limited to, a film coating of hydroxypropylcellulose (HPC), hydroxypropylmethylcellulose (HPMC), polyvinylpyrrolidone (PVP), polyethylene oxide (PEO), or other known pharmaceutical film coatings. In other embodiments, the delay coating can be a pressed coating of a placebo layer of various ingredients that erode at a measured rate to give the desired coating. Ingredients include, but are not limited to, at least one polymeric pharmaceutical excipient and/or at least one non-polymeric pharmaceutical excipient. The eroding coating may be in the form of a pressed powder coating comprising polymeric pharmaceutical excipients such as HPC, HPMC, PVP, microcrystalline cellulose, starch, and PEO, and/or non-polymeric pharmaceutical excipients such as lactose and sugars. In this preferred embodiment, a second reservoir of drug is contained in an outer annular sheath as described in U.S. application Ser. Nos. 10/419,536 entitled “Dosage Form with a Core Tablet of Active Ingredient Sheathed in a Compressed Angular Body of Powder or Granular Material, and Process and Tooling for Producing It,” filed on Apr. 21, 2003; and Ser. No. 10/379,338 entitled “Controlled Release Dosage Forms,” filed on Mar. 3, 2003, which are hereby incorporated by reference. The outer annular ring is formulated with the levodopa and carboxylase enzyme inhibitor and formulated for either immediate release or sustained release delivery for the desired time. Most preferably, the outer annular ring is formulated as a short duration controlled release formulation. The outer sheath comprises about 0 mg to 470 mg of levodopa, preferably about 100 to 150 mg of levodopa, and more preferably further comprises about 50 mg of carbidopa. In one most preferable embodiment, the inner delay coated tablet comprises about 50 mg of levodopa formulated to start drug release at least about 15 minutes after leaving the stomach and having a controlled release drug profile that releases drug over about three hours while the outer annular sheath comprises about 150 mg levodopa and 50 mg carbidopa having a controlled release drug profile that starts drug release immediately upon entering the stomach and releases drug over about three hours. In another most preferable embodiment, the inner delay coated tablet comprises about 100 mg levodopa formulated to start drug release at least about 15 minutes after leaving the stomach and having a controlled release drug profile that releases drug over about three hours while the outer annular sheath comprises about 100 mg levodopa and about 50 mg carbidopa having a controlled release drug profile that starts drug release immediately upon entering the stomach and releases drug over about three hours.

Optionally, the compositions of the above embodiments may further comprise at least one pharmaceutically acceptable organic acid. As used herein, the term “pharmaceutically acceptable organic acid” refers to a weak organic acid. Pharmaceutically acceptable organic acids suitable for use in the compositions of the invention include, but are not limited to, fumaric, citric, ascorbic, maleic, glutamic, malonic, gallic, tartaric, or oxalic acid. Preferred pharmaceutically acceptable organic acids include ascorbic acid, or citric acid.

Immediate release doses of the embodiments described above are formulated with standard excipients including, but not limited to, microcrystalline cellulose, starch, or lactose. Preferably, the immediate release dose further comprises a super-disintegrant. Superdisintegrants are disintegrants that expand upon contact with water. Examples of superdisintegrants include cross-linked carboxymethyl cellulose sodium (a.k.a. croscarmellose sodium), sodium starch glycolate, and cross-linked polyvinyl pyrollidone (a.k.a. crospovidone). Croscarmellose sodium is commercially available from FMC Corp. under the trade name Ac-Di-Sol® and from Avebe Corp. under the trade name Primellose®. Sodium starch glycolate is commercially available from Penwest Pharmaceuticals Co. under the tradename Explotab® and from Avebe Corp. under the tradename Primojel®. Crospovidone is commercially available from BASF Corp. under the tradename Kollidon® CL and from International Specialty Chemicals Corp. under the tradename Polyplasdone®. The most preferred superdisintegrants are croscarmellose sodium and crospovidone.

Controlled release embodiments are formulated using methods well known in the art. In the formulations of the invention, the dosage form comprises one or more excipients in an amount and grade to provide a controlled release profile of about 2 to about 10 hours, as desired. More preferably, the controlled release profile is about 2 to about 4 hours. One skilled in the art can determine the appropriate amount and grade of excipients required to achieve this release profile without undue experimentation.

In addition to the above-described excipients, the drug delivery vehicle may further include one or more other excipients that may be added to the vehicle for a variety of purposes. It will be understood by those in the art that some substances serve more than one purpose in a dosage form. For instance, some substances are binders that help hold a tablet together after compression, yet are disintegrants that help break the tablet apart once it reaches a patient's stomach.

Diluents increase the bulk of a solid pharmaceutical product and may make it easier for the patient and care giver to handle. Diluents include, but are not limited to, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, or talc.

Compacted dosage forms like those of the present invention may include excipients whose functions include helping to bind the active ingredient and other excipients together after compression. Binders for solid pharmaceutical compositions include, but are not limited to, acacia, alginic acid, carbomer (e.g., carbopol), carboxymethylcellulose sodium, dextrin, ethyl cellulose, gelatin, glucose, guar gum, hydrogenated vegetable oil, hydroxyethyl cellulose, hydroxypropyl cellulose (e.g., Klucel®), hydroxypropyl methylcellulose (e.g., Methocel®), liquid glucose, magnesium aluminum silicate, maltodextrin, methylcellulose, polymethacrylates, polyvinylpyrrolidone (e.g., Kollidon®, Plasdone®), starch, pregelatinized starch, sodium alginate, or alginate derivatives.

The dissolution rate of a compacted dosage form in the patient's stomach also may be adjusted by the addition of a disintegrant or second superdisintegrant to the dosage form, in addition to the superdisintegrant of the present inventive composition. Such additional disintegrants include, but are not limited to, alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), or starch.

Glidants can be added to improve the flow properties of a solid composition and improve the accuracy of dosing. Excipients that may function as glidants include, but are not limited to, colloidal silicon dioxide, magnesium trisilicate, powdered cellulose, starch, talc, or tribasic calcium phosphate.

When a dosage form such as a tablet is made by compaction, a composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease release of the product from the dye. Lubricants include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, surfactants, talc, waxes, or zinc stearate.

Flavoring agents and flavor enhancers make the dosage form more palatable to the patient. Common flavoring agents and flavor enhancers for pharmaceutical products that may be included in the drug delivery vehicle of the present invention include, but are not limited to, maltol, vanillin, ethyl vanillin, menthol, citric acid, fumaric acid ethyl maltol, or tartaric acid.

The dosage forms may also be colored using any pharmaceutically acceptable colorant to improve their appearance and/or facilitate patient identification of the product and unit dosage level.

The invention also encompasses methods of treating Parkinson's Disease comprising administering a safe and therapeutically effective amount of the compositions of the invention to a patient in need thereof. One method of treatment comprises administering about 30 mg to 500 mg of levodopa in a delayed release fashion such that the drug is essentially delivered after passage through the stomach and duodenum. In one embodiment, the method further comprises a reservoir that substantially does not release drug until after passing the duodenum releases the drug in the upper jejunum or about 10 to 40 minutes after passing from the stomach. In another embodiment, the drug is released in the mid or end of the jejunum or about 40 to 80 minutes after passing from the stomach. In yet another embodiment, the drug is released in the ileum or in the terminal ileum or about 80 to 240 minutes after passing from the stomach. In a more preferred embodiment, the drug is released in the upper or mid jejunum or about 10 to 60 minutes after passing from the stomach, and more preferably about 20 minutes after passing from the stomach. Preferably, the method of treatment comprises administering about 50 mg to about 200 mg of levodopa in a delayed dosage form. In preferred embodiments, the method encompasses also dosing at least one carboxylase enzyme inhibitor, wherein the carboxylase enzyme inhibitor is carbidopa or benserazide. The carboxylase enzyme inhibitor may be present in an amount of about 10 mg to about 100 mg per dose. In other preferred embodiments, the method further comprises dosing a COMT inhibitor such as entacapone. The COMT inhibitor may be present in amounts of about 50 mg to about 300 mg per dose, and most preferably about 200 mg.

More preferably, the method of treatment comprises a dose that starts releasing drug immediately and continues to do so for about 2 to 3 hours and a dose delayed for delivery for after passage through the stomach and the duodenum as described in the above embodiments. The dose that starts to release immediately contains about 50 mg to about 200 mg of levodopa, and most preferably contains about 150 mg of levodopa or 100 mg of levodopa. The delayed release dose of levodopa provided in the method of treatment is preferably about 50 mg to about 100 mg, and most preferably is about the amount needed to reach 200 mg levodopa in the combined dose. The composition is preferably administered in a dosage form providing a dose that starts releasing drug immediately and continues to do so for 2 to 3 hours and a dose delayed for delivery for after passage through the stomach and the duodenum as described in the above embodiments. The two doses in this method may be in separate dosage forms or be formulated in one dosage form. In another embodiment of this aspect of the invention, the invention comprises dosing levodopa in a delayed form so that the drug is dosed to the colon. In more preferred embodiments, this dose is given along with the doses previously described.

Having described the invention with reference to certain preferred embodiments, other embodiments will become apparent to one skilled in the art from consideration of the specification. The invention is further defined by reference to the following examples describing in detail the compositions of the invention. It will be apparent to those skilled in the art that many modifications, both to materials and methods, may be practiced without departing from the scope of the invention.

### EXAMPLES

### Example 1

### Enteric Coated Controlled Release Dosage Form Granulation

Levodopa (415 gm), anhydrous citric acid (60 gm), and Povidone (PVP K-30, 25 gm) were added to the bowl of the Diosna P(1/6) granulator. The mixture of powders was mixed at 380 rpm for 5 minutes. Ethanol 95% (22.5 gm) was added over 45 seconds while the mass was continued to be mixed at 380 rpm. The wet granulate was further massed at 380 rpm for another 45 seconds. The wet mass was discharged and transferred to a Diosna Mini Lab fluid bed dryer where it was dried at a fan set point of 45% and an inlet temperature of 30° C. to a volatiles level of <2%. A three gram sample was tested for loss on drying (LOD). The drying took about 30 minutes and yielded a dry granulate weighing 478.7 gm (96.4% yield).

The granulate was milled through a 0.63 mm screen in an erweka mill to give 475.4 grams of milled granulate (99.3% yield). A 25 gram sample was tested for particle size distribution and gave the following results:

| | | | | | | |
|---|---|---|---|---|---|---|
| Pore size(μ) | <75 | 75-150 | 150-250 | 250-355 | 355-500 | >500 |
| % of weight | 6.9 | 35.2 | 34.4 | 12.1 | 9.7 | 0.4 |

Tablet Blend and Pressing

The levodopa granulate from above (400 gm) was charged into a 2.5 liter V-mixer. Polyethylene glycol (PEG6000, 83 gm), Cellactose 80 (172.6 gm), and Povidone (PVP K-30, 166 gm) were added and mixed for 5 minutes. Magnesium stearate (8.3 gm) was added and the blend mixed for another 30 seconds. The blend weighed 823.8 grams. The blend was pressed into tablets weighing 500±25 mg using a Kilian RTS 20 tablet press and 11 mm diameter normal concave punches. The tablets formed had the following properties:

| Avg wgt | | Avg tablet | | Avg tablet | |
|---|---|---|---|---|---|
| (mg) | % RSD | hardness (Kp) | % RSD | thickness (mm) | % RSD |
| 508.2 | 0.51 | 12.9 | 3.5 | 5.30 | 0.44 |

Enteric Coating

Ethanol 95% (940 gm) was added to an appropriate vessel equipped with a magnetic stirrer. Eudragit L-100 (60 gm) was added and stirred until all dissolved. Triethylcitrate (12 gm) was added and the mixture stirred overnight until complete dissolution of all components.

Levodopa tablets (521.3 gm) were charged into a HI Coater perforated pan coater and preheated to 30-32° C. at a tumbling rate of 15 rpm. The tablets were coated using the following parameters:

| Parameter | Value |
|---|---|
| Inlet air temperature | 45° C. |
| Outlet air temperature | 28.8° C. |
| Differential Air flow | 596 mm³/min |
| Pan rotation | 13 rpm |
| Bed temperature | 28.8° C. |
| Air ressure in die | 0.6 bar |

The coating was continued until the tablets had gained on the average 22 mg coating. The coating process took about 3 hours.
In Vitro Drug Release Tests

The tablets were tested for two hours for drug release in 900 ml 0.1N HCl at 37° C. and 50 rpm using a USP type II dissolution bath. Drug content was determined by HPLC using the following conditions:

Column: 250×4.6 mm, 5μ Hypersil BDS C18

Mobile phase: 2.5% acetonitrile and 97.5% 0.1N phosphate buffer pH=2.8

Flow rate and retention time: 1.2 ml/min, 4.0 min

Detector parameters: UV at 280 nm

The tablets showed the following release in acid conditions:

| | | | | | | |
|---|---|---|---|---|---|---|
| Tablet # | 1 | 2 | 3 | 4 | 5 | 6 |
| % release at 2 hrs | 2.32 | 1.61 | 0.61 | 1.92 | 0.33 | 1.27 |
| Tablet # | 7 | 8 | 9 | 10 | 11 | 12 |
| % release at 2 hrs | 0.35 | 0.95 | 0.09 | 1.67 | 1.66 | 0.55 |

The tablets were tested for six more hours for drug release in 900 ml 0.1 N phosphate buffer of pH=6.8 at 37° C. and 50 rpm using a USP type II dissolution bath and analyzed by HPLC using the same conditions as above. The results of the drug release were as follows:

| Time (hr) | Average % cumulative release | % RSD |
|---|---|---|
| 3 | 18.5 | 39.6 |
| 4 | 52.5 | 23.4 |
| 5 | 75.9 | 18.7 |
| 6 | 90.5 | 12.0 |
| 7 | 96.2 | 7.7 |
| 8 | 96.0 | 4.4 |

### Example 2

### Pharmacokinetic Trial of the Dosage Form of Example 1

A pharmacokinetic trial of the delayed release levodopa formulation from example 1 was carried out in healthy volunteers. Two parallel trials were run. One was of dosing in the morning—daytime dosing while the other was with dosing before bedtime—nighttime dosing. The study synopsis is presented in Table 1

TABLE 1
Study Synopsis

| | |
|---|---|
| STUDY TITLE | A Single-Dose, Dual-Group (Daytime vs. Nighttime Dosing), |
| | Two-Way Crossover Comparative Bioavailability Study of |
| | Levodopa, Between A Novel Enteric-Coated, Delayed |
| | Controlled-Release Test Formulation of Levodopa (200 mg; Test |
| | Sample) in Combination with Immediate-Release Lodosyn ® (2 × |
| | 25 mg Carbidopa; Merck & Co., Inc.) versus Sinemet-CR ® |
| | (Levodopa/Carbidopa 200/50 mg; Merck & Co., Inc.) + |
| | Comtan ® (Entacapone 200 mg; Orion), in 24 Healthy Male |
| | Volunteers |
| TEST DRUGS | 1 × Levodopa Enteric-Coated, Delayed Controlled-Release Test |
| | Tablet (200 mg; Teva R&D Initiative) + 2 × Lodosyn ® tablets |
| | (carbidopa, 25 mg; Merck & Co., Inc.) |
| REFERENCE DRUGS | 1 × Sinemet-CR ® tablet (Levodopa/Carbidopa 200/50 mg; Merck |
| | & Co., Inc.) + |
| | 1 × Comtan ® tablet (200 mg Entacopone, 200 mg; Orion) |
| PRE-STUDY DRUG | Lodosyn ® (carbidopa, 25 mg; Merck & Co., Inc.) 2 × 25 mg |
| | tablets (50 mg dose), three times daily, for two consecutive days |
| | prior to each study session. |
| DOSAGE FORM | Tablets |
| MODE OF | Oral |
| ADMINISTRATION | |
| PRIMARY | (1) To compare the pharmacokinetic profiles |
| OBJECTIVES | (Cₘₐₓ, Tₘₐₓ, AUC_{T}, AUC₁) |
| | obtained for levodopa and carbidopa, following |
| | daytime oral administration of a single-dose of the test |
| | formulation at a unit dose of 200 mg levodopa (to be |
| | administered in combination with immediate-release |
| | carbidopa, 50 mg) as compared to the |
| | pharmacokinetic profiles (Cₘₐₓ, |
| | Tₘₐₓ, AUC_{T}, AUC_{I}) obtained for levodopa and |
| | carbidopa, following daytime oral ingestion of |
| | a single-dose of the reference formulation, |
| | Sinemet-CR ®, at a unit dose of 200 |
| | mg levodopa (in combination with carbidopa 50 mg) plus |
| | Comtan ® (entacapone 200 mg). |
| | (2) To compare the pharmacokinetic profiles |
| | (Cₘₐₓ, Tₘₐₓ, AUC_{T}, |
| | AUC_{I}) obtained for levodopa and carbidopa, following |
| | nighttime oral administration of a single-dose of the test |
| | formulation at a unit dose of 200 mg levodopa (to be |
| | administered in combination with immediate-release |
| | carbidopa, 50 mg) as compared to the |
| | pharmacokinetic profiles |
| | (Cₘₐₓ, Tₘₐₓ, AUC_{T}, AUC_{I}) obtained for levodopa and |
| | carbidopa, following nighttime oral ingestion of a |
| | single-dose of the reference |
| | formulation, Sinemet-CR ®, at a unit dose of |
| | 200 mg levodopa (in combination with carbidopa |
| | 50 mg) plus Comtan ® (entacapone 200 mg). |
| SECONDARY | To monitor the subjects for safety (adverse |
| OBJECTIVES | events, vital signs and changes in laboratory tests) during the |
| | study period and to correlate |
| | adverse effects (if apparent) to pharmacokinetic parameters. |
| NUMBER OF | 24 healthy males, to be divided into 2 groups, |
| SUBJECTS: | “A” and “B”, with all subjects in the “A” group to |
| | be dosed (Test vs. Reference) in the |
| | daytime, and all subjects in the “B” group to |
| | be dosed (Test vs. |
| | Reference) at nighttime. Regardless of dosing time, |
| | however, all subjects in both groups will |
| | be treated the same way, i.e., receive carbidopa pre-dosing for |
| | 2 days prior to dosing, be fasted for at |
| | least 10 hours prior to dosing, and be followed pharmacokinetically |
| | and monitored for safety for 24 hours following initial dosing. |
| MAIN INCLUSION | Healthy Caucasian male volunteers aged 18-55 years who have |
| CRITERIA: | provided written informed consent |
| STUDY | Pre-treatment: 2 × 25 mg carbidopa (Lodosyn ®, Merck & Co., Inc.) |
| ADMINISTRATIONS: | three times daily - 2 days |
| | Administration 1 (A): 1 × 200 mg Enteric-Coated, Delayed |
| | Controlled-Release Levodopa TEST tablet (Test) + 2 × 25 mg |
| | Lodosyn ® (Carbidopa/Merck & Co., Inc.) tablets, for a total unit |
| | dose of 200 mg LD (Test) and 50 mg CD, to be administered as |
| | three (3) separate tablets ingested simultaneously; Test |
| | Administration 2 (B): 1 × Sinemet-CR ® tablet |
| | (Levodopa/Carbidopa 200/50 mg; Merck & Co., Inc.) + Comtan ® |
| | (Entacapone, 200 mg; Orion), to be administered as two (2) |
| | separate tablets ingested simultaneously; Reference |
| STUDY DESIGN | Randomized, open label, dual-group (daytime vs. nighttime |
| | dosing), two-way, two period, comparative crossover study. |
| | For both groups (daytime vs. nighttime dosing), the wash-out |
| | period between study sessions will be at least 2 weeks. |
| BLOOD SAMPLING | For both groups (daytime vs. nighttime dosing): 0″ hour (pre- |
| | dosing), 30 minutes, 1.0, 2.0, 3.0, 4.0, 5.0, 6.0, 7.0, 8.0, 9.0, 10.0, |
| | 11.0, 12.0, 16.0, and 24.0 hours post-dose (in-house), for a total of |
| | 16 blood samples per study period. |
| CRITERIA FOR | Pharmacokinetic parameters (Cₘₐₓ, Tₘₐₓ, AUC) determined from |
| EVALUATION | plasma concentrations of levodopa and carbidopa following oral |
| | administration of each of the drug administrations: Test and |
| | Reference. |
| ANALYTICAL | All plasma samples will be analyzed for levodopa and carbidopa |
| METHOD USED | levels using a validated LC/MS method |
| DURATION OF | Up to 7 weeks (including screening period) |
| STUDY (CLINICAL | |
| PHASE) | |

Results of Daytime Dosing:

The raw data that was obtained for the concentration of levodopa in plasma for daytime dosing of both the test and the reference formulations are presented in Tables 2A and 2B.

TABLE 2A
Raw data for levodopa in plasma (ng/ml) for test formulation daytime dosing

| (Day:hr: | | | | | | | | | | | | |
|---|---|---|---|---|---|---|---|---|---|---|---|---|
| Min) | 01 | 02 | 03 | 04 | 05 | 06 | 07 | 08 | 09 | 10 | 11 | 12 |
| 01:00:00 | BLQ | BLQ | BLQ | 73.82 | BLQ | BLQ | BLQ | BLQ | BLQ | BLQ | BLQ | BLQ |
| 01:00:30 | BLQ | BLQ | BLQ | BLQ | BLQ | 37.75 | BLQ | BLQ | BLQ | BLQ | BLQ | BLQ |
| 01:01:00 | 228.96 | 36.45 | BLQ | BLQ | 46.46 | 68.69 | BLQ | 289.34 | 188.38 | BLQ | BLQ | BLQ |
| 01:02:00 | 55.86 | 51.89 | BLQ | BLQ | 63.25 | BLQ | 33.86 | BLQ | BLQ | BLQ | BLQ | 335.42 |
| 01:03:00 | 919.96 | 324.46 | BLQ | 369.48 | 514.76 | 105.46 | 871.53 | 361.64 | BLQ | 798.73 | BLQ | 1678.44 |
| 01:04:00 | 1077.61 | 1523.70 | BLQ | 682.26 | 1643.50 | 103.76 | 805.77 | 1340.45 | 100.18 | 1488.35 | 667.66 | 1095.69 |
| 01:05:00 | 543.91 | 728.73 | 1232.33 | 360.33 | 643.41 | 79.49 | 307.61 | 553.71 | 797.80 | 990.29 | 1807.35 | 835.44 |
| 01:06:00 | 255.34 | 395.83 | 2100.73 | 176.72 | 327.15 | 50.64 | 185.95 | 301.88 | 649.52 | 578.15 | 710.96 | 710.96 |
| 01:07:00 | 141.16 | 235.81 | 612.46 | 102.64 | 193.78 | 178.99 | 84.17 | 188.47 | 463.69 | 340.98 | 503.51 | 342.88 |
| 01:08:00 | 87.62 | 168.11 | 325.22 | 57.40 | 107.12 | BLQ | 62.59 | 129.74 | 269.24 | 208.29 | 297.35 | 325.41 |
| 01:09:00 | 61.43 | 94.72 | 200.43 | 33.34 | 66.31 | BLQ | 53.01 | 86.43 | 152.51 | 115.60 | 208.62 | 114.45 |
| 01:10:00 | 52.11 | 60.45 | 131.63 | BLQ | 35.64 | BLQ | 29.88 | 57.89 | 115.03 | 150.33 | 122.39 | 63.09 |
| 01:11:00 | 33.19 | 39.97 | 81.12 | BLQ | BLQ | BLQ | BLQ | 53.54 | 75.46 | 52.57 | 160.09 | 58.76 |
| 01:12:00 | BLQ | BLQ | 54.89 | BLQ | BLQ | BLQ | BLQ | 34.46 | 66.83 | 116.89 | 882.41 | 158.13 |
| 01:16:00 | 104.87 | 854.22 | BLQ | BLQ | BLQ | 597.25 | BLQ | 267.05 | BLQ | BLQ | 623.18 | 714.90 |
| 02:00:00 | 330.44 | 44.85 | BLQ | BLQ | BLQ | 53.58 | BLQ | 83.88 | BLQ | BLQ | 141.31 | 288.49 |

BLQ - Below the limit of quantitation which is 29.63 ng/ml.

TABLE 2B
Raw data for levodopa in plasma (ng/ml) for reference formulation daytime dosing

| (Day:hr: | | | | | | | | | | | | |
|---|---|---|---|---|---|---|---|---|---|---|---|---|
| Min) | 01 | 02 | 03 | 04 | 05 | 06 | 07 | 08 | 09 | 10 | 11 | 12 |
| 01:00:00 | BLQ | BLQ | BLQ | BLQ | BLQ | BLQ | BLQ | BLQ | 173.59 | BLQ | BLQ | BLQ |
| 01:00:30 | 134.57 | 77.52 | 241.49 | 387.08 | 55.96 | 1238.85 | 87.52 | 400.61 | 106.95 | 99.88 | 481.27 | 930.18 |
| 01:01:00 | 745.69 | 621.79 | 1349.15 | 1159.36 | 435.70 | 978.07 | 606.14 | 459.55 | 168.69 | 668.75 | 765.60 | 1676.93 |
| 01:02:00 | 1271.15 | 863.17 | 814.78 | 1293.80 | 848.61 | 1111.24 | 793.74 | 634.17 | 1512.48 | 1246.83 | 804.61 | 1012.26 |
| 01:03:00 | 552.77 | 770.28 | 620.12 | 1038.38 | 659.29 | 896.10 | 718.37 | 759.91 | 608.56 | 1171.08 | 846.39 | 487.13 |
| 01:04:00 | 791.97 | 530.60 | 860.10 | 1051.77 | 703.28 | 505.03 | 692.16 | 534.45 | 576.16 | 1142.33 | 815.21 | 567.73 |
| 01:05:00 | 468.99 | 310.16 | 528.78 | 712.75 | 485.18 | 334.18 | 334.08 | 757.77 | 882.63 | 696.24 | 659.02 | 933.83 |
| 01:06:00 | 247.78 | 193.43 | 296.99 | 422.63 | 309.90 | 235.14 | 275.03 | 431.53 | 960.39 | 581.47 | 348.40 | 591.12 |
| 01:07:00 | 177.92 | 116.54 | 181.29 | 307.61 | BLQ | 127.78 | 126.46 | 270.09 | 599.42 | 249.26 | 374.9 | 328.67 |
| 01:08:00 | 105.51 | 95.60 | 144.20 | 207.87 | 113.81 | 66.29 | 77.88 | 151.95 | 408.60 | 175.60 | 143.36 | 228.01 |
| 01:09:00 | 75.07 | 54.46 | 68.99 | 108.68 | 67.19 | 55.41 | 45.85 | 120.32 | 227.17 | 128.86 | 176.84 | 155.2 |
| 01:10:00 | 80.80 | 41.28 | 59.71 | 83.47 | 59.37 | BLQ | 33.03 | 80.35 | 173.77 | 68.10 | 131.8 | 136.13 |
| 01:11:00 | 40.45 | 34.59 | 68.55 | 50.70 | 29.82 | 35.30 | 62.35 | 64.42 | 130.59 | 48.84 | 52.57 | 82.90 |
| 01:12:00 | 39.67 | BLQ | 114.68 | 69.42 | BLQ | BLQ | BLQ | 45.10 | 86.38 | 31.45 | 60.82 | 69.69 |
| 01:16:00 | BLQ | BLQ | 115.93 | 50.57 | 47.12 | BLQ | 153.10 | BLQ | 189.70 | 189.50 | BLQ | BLQ |
| 02:00:00 | BLQ | BLQ | 170.39 | 137.34 | 684.17 | BLQ | BLQ | BLQ | 65.48 | 35.20 | BLQ | BLQ |

BLQ - Below the limit of quantitation which is 29.63 ng/ml

The pharmacokinetic parameters that were calculated from the raw data for both the test and reference formulation are presented in Table 3. Table 3 illustrated that the geometric mean of the area under the plasma concentration of levodopa vs. time curve (AUC) for the test formulation was about 86% of the reference and not an insignificant fraction thereof as might have been expected. The geometric mean of the ratio of the individual values (where each volunteer was his own control) was 86% of the reference. The geometric mean of the maximal concentration of the plasma concentration of levodopa vs. time curve (Cₘₐₓ) was slightly higher for the test formulation being about 110% of the reference. The geometric mean of the ratio of the individual values (where each volunteer was his own control) was 110% of the reference. The geometric mean elimination half life calculated for each of the two formulations was similar being 1.8 hours for the test formulation and 2.0 hours for the reference. The time to Cₘₐₓ(Tₘₐₓ) was different for the two formulations. The test formulation, which was designed to not allow drug release in the stomach or in the duodenum, had a geometric mean Tₘₐₓ of 4.1 hours compared to 1.7 hours for the reference which released its drug starting in the stomach.

One can conclude that the test formulation delivered its drug load beyond the duodenum to the jejunum and ileum. The raw data showed that in several of the volunteers some of the drug was absorbed between 12 and 24 hours indicating absorption from the colon in these individuals. Table 3 summarizes the pharmacokinetic parameters for Daytime dosing.

TABLE 3
Results of LD from LD-CD-SR-Day (200 mg dose)

| Vol.^{a} | | AUC (h*ng/g) | | AUC_{inf} (h*ng/g) | | t_{1/2} | | Tₘₐₓ (h) | | Cₘₐₓ (ng/g) | | Cₘₐₓtest/ | AUC_{test/} |
|---|---|---|---|---|---|---|---|---|---|---|---|---|---|
| Test | Ref | Test | Ref | Test | Ref | Test | Ref | Test | Ref | Test | Ref | Cₘₐₓref | AUC_{ref} |
| 1-1 | 1-2 | 5350.9 | 4538.1 | 6424.9 | 4538.1 | 2.3 | 2.7 | 4.0 | 2.0 | 1077.6 | 1271.2 | 0.85 | 1.18 |
| 2-1 | 2-2 | 8955.7 | 3515.2 | 9050.0 | 3515.2 | 1.5 | 2.1 | 4.0 | 2.0 | 1523.7 | 863.2 | 1.77 | 2.55 |
| 3-2 | 3-1 | 4821.1 | 6440.0 | 4821.1 | 6956.3 | 1.6 | — | 6.0 | 1.0 | 2100.7 | 1349.2 | 1.56 | 0.75 |
| 4-2 | 4-1 | 1800.6 | 7367.1 | 1800.6 | 7672.5 | 1.2 | 1.5 | 4.0 | 2.0 | 682.3 | 1293.8 | 0.53 | 0.24 |
| 5-2 | 5-1 | 3629.8 | 6650.6 | 3629.8 | 8723.8 | 1.2 | — | 4.0 | 2.0 | 1643.5 | 848.6 | 1.94 | 0.55 |
| 6-1 | 6-2 | 4386.6 | 4719.4 | — | 4719.4 | — | 1.7 | — | 0.5 | 597.3 | 1238.9 | 0.48 | 0.93 |
| 7-2 | 7-1 | 2434.4 | 4575.9 | 2434.4 | 4575.9 | 2.1 | 1.5 | 3.0 | 2.0 | 871.5 | 793.7 | 1.10 | 0.53 |
| 8-1 | 8-2 | 5314.7 | 4462.7 | 5314.7 | 4462.7 | 2.2 | 2.4 | 4.0 | 3.0 | 1340.5 | 759.9 | 1.76 | 1.19 |
| 9-2 | 9-1 | 2931.8 | 7919.2 | 2931.8 | 8117.7 | 2.6 | — | 5.0 | 2.0 | 797.8 | 1512.5 | 0.53 | 0.37 |
| 10-2 | 10-1 | 5015.5 | 7416.5 | 5015.5 | 7523.2 | 1.8 | — | 4.0 | 2.0 | 1488.4 | 1246.8 | 1.19 | 0.68 |
| 11-2 | 11-1 | 10988.3 | 5320.0 | 11416.5 | 5320.0 | — | — | 5.0 | 3.0 | 1807.4 | 846.4 | 2.14 | 2.07 |
| 12-2 | 12-1 | 11399.2 | 6420.0 | 11399.2 | 6420.0 | 2.1 | 2.3 | 3.0 | 1.0 | 1678.4 | 1676.9 | 1.00 | 1.78 |

| | | | | | | | | | | | | |
|---|---|---|---|---|---|---|---|---|---|---|---|---|
| Average | 5585.7 | 5778.7 | 5839.9 | 6045.4 | 1.8 | 2.0 | 4.2 | 1.9 | 1300.7 | 1141.8 | 1.236 | 1.067 |
| Geomean | 4821.5 | 5610.7 | 4966.6 | 5814.4 | 1.8 | 2.0 | 4.1 | 1.7 | 1208.7 | 1103.2 | 1.096 | 0.859 |
| stddev | 3190 | 1432 | 3404 | 1729 | 0.46 | 0.46 | 0.87 | 0.74 | 486.89 | 308.21 | 0.58 | 0.72 |
| % CV | 57.11 | 24.79 | 58.30 | 28.60 | 24.87 | 22.87 | 20.90 | 39.59 | 37.43 | 26.99 | | |

^{a}Volunteer - session

Results of Nighttime Dosing:

The raw data that was obtained for the concentration of levodopa in plasma for nighttime dosing of both the test and the reference formulations are presented in Tables 4A and 4B.

TABLE 4A
Test formulation - raw data for levodopa in plasma (ng/ml) for nighttime dosing

| (Day:Hour: | | | | | | | | | | | | |
|---|---|---|---|---|---|---|---|---|---|---|---|---|
| Min) | 13 | 14 | 15 | 16 | 17 | 18 | 19 | 20 | 21 | 22 | 23 | 24 |
| 01:00:00 | BLQ | 44.13 | BLQ | BLQ | 37.89 | BLQ | 83.73 | BLQ | BLQ | BLQ | 31.03 | 57.80 |
| 01:00:30 | BLQ | 29.84 | BLQ | 67.27 | BLQ | BLQ | BLQ | BLQ | BLQ | BLQ | BLQ | BLQ |
| 01:01:00 | BLQ | 39.17 | BLQ | 74.33 | 515.06 | 64.28 | BLQ | BLQ | BLQ | BLQ | 1561.09 | BLQ |
| 01:02:00 | 352.55 | 204.44 | BLQ | 279.33 | 48.51 | 79.06 | BLQ | BLQ | BLQ | BLQ | 69.60 | BLQ |
| 01:03:00 | 65.08 | 229.64 | 192.45 | 2722.13 | 379.00 | 51.92 | 30.52 | 35.89 | BLQ | BLQ | 1970.88 | BLQ |
| 01:04:00 | 1013.10 | 947.67 | 288.20 | 956.30 | 411.40 | 1184.81 | 65.56 | 600.12 | BLQ | BLQ | 1441.34 | BLQ |
| 01:05:00 | 2392.23 | 1857.46 | 1699.98 | 480.85 | 1799.39 | 2062.13 | 182.65 | 1033.71 | 975.98 | 1874.21 | 631.78 | BLQ |
| 01:06:00 | 941.61 | 905.40 | 883.16 | 297.32 | 1694.49 | 415.33 | 605.54 | 440.11 | 1528.34 | 1365.89 | 316.69 | 1635.18 |
| 01:07:00 | 489.39 | 550.87 | 461.29 | 220.18 | 585.88 | 771.85 | 1504.16 | 318.91 | 672.04 | 823.34 | 199.79 | 1565.02 |
| 01:08:00 | 324.48 | 337.86 | 497.05 | 216.12 | 652.69 | 261.71 | 752.66 | 138.24 | 892.61 | 404.54 | 120.92 | 1140.83 |
| 01:09:00 | 148.44 | 267.95 | 164.07 | 110.87 | 437.37 | 259.65 | 266.99 | 99.13 | 386.43 | 258.25 | 83.82 | 336.44 |
| 01:10:00 | 95.30 | 144.60 | 113.65 | 53.76 | 1075.62 | 229.68 | 242.38 | 72.50 | 941.03 | 128.63 | 35.19 | 169.70 |
| 01:11:00 | 103.06 | 79.55 | 60.72 | 57.83 | 93.32 | 129.24 | 128.86 | 36.97 | 119.60 | 190.44 | BLQ | 282.52 |
| 01:12:00 | 101.99 | 61.19 | 105.41 | BLQ | 60.98 | 67.51 | 94.89 | BLQ | 71.34 | 63.62 | BLQ | 49.51 |
| 01:16:00 | BLQ | BLQ | 282.60 | BLQ | BLQ | BLQ | 779.75 | BLQ | BLQ | BLQ | BLQ | BLQ |
| 02:00:00 | BLQ | BLQ | BLQ | BLQ | 177.49 | BLQ | BLQ | BLQ | BLQ | BLQ | BLQ | BLQ |

BLQ - Below the limit of quantitation 29.63 ng/ml.

TABLE 4B
Reference formulation - raw data for levodopa in plasma (ng/ml) for nighttime dosing

| (Day:hr: | | | | | | | | | | | | |
|---|---|---|---|---|---|---|---|---|---|---|---|---|
| Min) | 13 | 14 | 15 | 16 | 17 | 18 | 19 | 20 | 21 | 22 | 23 | 24 |
| 01:00:00 | 265.65 | BLQ | 121.27 | BLQ | BLQ | 56.46 | BLQ | BLQ | 980.97 | BLQ | BLQ | BLQ |
| 01:00:30 | 56.24 | 319.63 | 244.39 | 307.86 | BLQ | 168.35 | 42.27 | 59.32 | 214.54 | 283.89 | 145.81 | 120.78 |
| 01:01:00 | 199.84 | 580.74 | 711.11 | 745.84 | 146.31 | 1007.81 | 1134.43 | 604.20 | 555.75 | 1143.49 | 149.62 | 149.46 |
| 01:02:00 | 398.12 | 1445.84 | 325.37 | 1213.42 | 233.82 | 465.62 | 1258.59 | 1202.31 | 832.77 | 1504.32 | 205.60 | 232.02 |
| 01:03:00 | 505.13 | 1359.93 | 972.36 | 1049.50 | 2280.41 | 1109.79 | 928.81 | 1050.64 | 1570.54 | 1475.21 | 368.40 | 1073.57 |
| | | | | | κ | | | | | | | |
| 01:04:00 | 617.16 | 1180.36 | 646.56 | 989.86 | 1587.75 | 1262.61 | 1020.22 | 813.15 | 1147.77 | 990.42 | 716.97 | 688.74 |
| 01:05:00 | 1545.97 | 847.82 | 588.97 | 1147.27 | 1293.97 | 1236.43 | 795.99 | 1077.89 | 749.13 | 710.76 | 1482.14 | 357.65 |
| 01:06:00 | 1122.29 | 560.48 | 1299.24 | 365.14 | 1083.2 | 588.45 | 548.67 | 710.32 | 511.41 | 466.85 | 1511.24 | 606.36 |
| 01:07:00 | 1270.90 | 400.61 | 697.96 | 393.13 | 764.68 | 966.15 | 297.23 | 443.97 | 349.56 | 288.89 | 724.89 | 331.44 |
| 01:08:00 | 614.14 | 268.81 | 424.28 | 289.61 | 675.36 | 377.23 | 215.78 | 336.84 | 249.41 | 207.24 | 514.98 | 150.77 |
| 01:09:00 | 409.01 | 383.22 | 308.26 | 167.09 | 529.43 | 113.75 | 141.38 | 195.85 | 138.34 | 149.31 | 296.81 | 90.75 |
| 01:10:00 | 277.39 | 112.35 | 186.28 | 123.55 | 235.96 | 356.40 | 62.93 | 121.07 | 105.38 | 100.70 | 195.84 | 66.85 |
| 01:11:00 | 198.44 | 251.27 | 95.41 | 84.81 | 148.36 | 200.56 | 40.27 | 96.02 | 63.63 | 49.65 | 1627.54 | 30.46 |
| 01:12:00 | 1377.11 | 66.08 | 62.90 | 45.40 | 145.87 | 163.89 | 40.04 | 57.77 | 46.51 | BLQ | 75.08 | BLQ |
| 01:16:00 | BLQ | BLQ | BLQ | BLQ | BLQ | 102.09 | BLQ | 1563.49 | BLQ | BLQ | BLQ | BLQ |
| 02:00:00 | BLQ | 82.22 | BLQ | BLQ | BLQ | BLQ | BLQ | BLQ | BLQ | BLQ | BLQ | BLQ |

BLQ - Below the limit of quantitation 29.63 ng/ml.
k - Value calculated by extrapolating standard curve.

The pharmacokinetic parameters that were calculated from the raw data for both the test and reference formulation are presented in Table 5. The geometric mean of the AUC for the test formulation was about 75% of that of the reference and not an insignificant fraction thereof as might have been expected. The value for the test formulation was about 20% higher than for daytime dosing while the value for the reference was about 44% higher for the nighttime dosing than for the daytime dosing. The geometric mean of the ratio of the individual values for the nighttime dosing (where each volunteer was his own control) was also 75%. The geometric mean of the Cₘₐₓ was higher for the test formulation being about 124% of the reference. The geometric mean of the ratio of the individual values (where each volunteer was his own control) was 124%. The Cₘₐₓ for nighttime dosing was 48% higher than daytime dosing for the test formulation and 31% higher for the reference formulation. The geometric mean elimination half life calculated for each of the two formulations was shorter than the daytime dosing values being 1.3 hours for the test formulation and 1.5 hours for the reference. The Tₘₐₓ for nighttime dosing shows less of a difference than was seen for daytime dosing. The test formulation, which was designed to not allow drug release in the stomach or in the duodenum, has a geometric mean Tₘₐₓ of 4.9 hours compared to 3.3 hours for the reference. Table 5 illustrates the pharmacokinetic parameters of levodopa for nighttime dosing.

TABLE 5
Results of LD from LD-CD-SR-Day (200 mg dose)

| Vol.^{a} | | AUC (h*ng/g) | | AUC_{inf}(h*ng/g) | | t_{1/2} | | Tₘₐₓ(h) | | Cₘₐₓ (ng/g) | | Cₘₐₓtest/ | AUCₜₑₛₜ/ |
|---|---|---|---|---|---|---|---|---|---|---|---|---|---|
| Test | Ref | Test | Ref | Test | Ref | Test | Ref | Test | Ref | Test | Ref | Cₘₐₓref | AUC_{ref} |
| 1-1 | 1-2 | 6180.2 | 10645.7 | 6180.2 | 10645.7 | | | 5.0 | 5.0 | 2392.2 | 1546.0 | 1.55 | 0.58 |
| 2-2 | 2-1 | 5733.7 | 7900.1 | 5733.7 | 7900.1 | 1.4 | | 5.0 | 2.0 | 1857.5 | 1445.8 | 1.28 | 0.73 |
| 3-2 | 3-1 | 6319.7 | 6387.8 | 6319.7 | 6387.8 | | 1.3 | 5.0 | 6.0 | 1700.0 | 1299.2 | 1.31 | 0.99 |
| 4-1 | 4-2 | 5484.1 | 6650.2 | 5484.1 | 6650.2 | 1.0 | 1.4 | 3.0 | 2.0 | 2722.1 | 1213.4 | 2.24 | 0.82 |
| 5-1 | 5-2 | 8435.8 | 9306.9 | 8968.9 | 9306.9 | | 1.6 | 5.0 | 3.0 | 1799.4 | 2280.0 | 0.79 | 0.91 |
| 6-2 | 6-1 | 5662.4 | 8553.4 | 5662.4 | 8553.4 | 1.1 | 3.8 | 5.0 | 4.0 | 2062.1 | 1262.6 | 1.63 | 0.66 |
| 7-1 | 7-2 | 8716.0 | 6281.9 | 8716.0 | 6281.9 | | 1.5 | 7.0 | 2.0 | 1504.2 | 1258.6 | 1.20 | 1.39 |
| 8-2 | 8-1 | 2775.6 | 16056.2 | 2775.6 | 16056.2 | 1.6 | | 5.0 | | 1033.7 | 1563.5 | 0.66 | 0.17 |
| 9-2 | 9-1 | 5694.4 | 6603.5 | 5694.4 | 6603.5 | | 1.7 | 6.0 | 3.0 | 1528.3 | 1570.5 | 0.97 | 0.86 |
| 10-2 | 10-1 | 5204.4 | 6942.9 | 5204.4 | 6942.9 | 1.7 | 1.0 | 5.0 | 2.0 | 1874.2 | 1504.3 | 1.25 | 0.75 |
| 11-2 | 11-1 | 6048.6 | 8017.2 | 6048.6 | 8017.2 | 1.2 | | 3.0 | 11.0 | 1970.9 | 1627.5 | 1.21 | 0.75 |
| 12-2 | 12-1 | 5267.9 | 3801.1 | 5267.9 | 3801.1 | | 1.2 | 6.0 | 3.0 | 1635.2 | 1073.6 | 1.52 | 1.39 |

| | | | | | | | | | | | | |
|---|---|---|---|---|---|---|---|---|---|---|---|---|
| Average | 5960.2 | 8095.6 | 6004.6 | 8095.6 | 1.4 | 1.7 | 5.0 | 3.9 | 1840.0 | 1470.4 | 1.301 | 0.833 |
| Geomean | 5762.3 | 7648.5 | 5791.8 | 7658.5 | 1.3 | 1.5 | 4.9 | 3.3 | 1791.8 | 1444.3 | 1.241 | 0.753 |
| stddev | 1524 | 3043 | 1608 | 3043 | 0.28 | 0.89 | 1.13 | 2.70 | 434.25 | 308.54 | 0.42 | 0.33 |
| %CV | 25.56 | 37.59 | 26.78 | 37.59 | 21.06 | 52.70 | 22.56 | 69.07 | 23.60 | 20.98 | | |

^{a}Volunteer - session

The value of 3.3 hours was obtained despite the fact that drug release in the reference commences in the stomach. The raw data showed that in several of the volunteers some of the drug was absorbed between 12 and 24 hours indicating absorption in the colon. It would seem that the difference in the effect between daytime and nighttime dosing on Tₘₐₓ was greater for the reference than for the test formulation. The test formulation was delayed from 4.1 hours to 4.9 hours (an approximately 20% delay) while the reference formulation the geometric mean of the Tₘₐₓ was delayed from 1.7 hours to 3.3 hours (an approximately 96% delay).

### Example 3

### Combination Tablets with an Oblong Contour Granulation

Levodopa (470 gm) and Povidone (PVP K-30, 30 gm) were added to the bowl of a Diosna P(1/6) granulator. The mixture of powders was mixed at 380 rpm for 5 minutes. Ethanol 95% (35 ml) was added over 30 seconds while the mass was mixed at 380 rpm. The wet granulate was further massed at 380 rpm for another 30 seconds. The wet mass was discharged and transferred to an Aeromatic Lab fluid bed dryer where it was dried at an inlet temperature of 30° C. to a volatiles level of 1.2%. The granulate was milled through a 0.63 mm screen in an erweka mill to give milled granulate.

The levodopa granulate from above (400 gm) was charged into a 2.5 liter V-mixer. Cellactose 80 (292 gm) and hydroxpropylmethylcellulose (Methocel K15M, 53 gm) were added and mixed for 5 minutes. Magnesium stearate (7 gm) was added and the blend mixed for another 30 seconds. The blend weighed 752 grams. The blend was pressed into tablets weighing 200 mg using a Manesty Express tablet press with oblong 6.35 mm×10.5 mm punches. The tablets formed had the following properties:

| Tablet wgt (mg) | tablet hardness (Kp) | tablet thickness (mm) |
|---|---|---|
| 200 | 8-11 | 2.7-2.8 |

Enteric Coating

Purified water (435 gm) was added to an appropriate vessel equipped with a magnetic stirrer. Eudragit L30 D55 (533 gm) was added and stirred until a uniform suspension was obtained. Triethylcitrate (16 gm) was added and the mixture stirred until uniform. Levodopa tablets (600 gm) were charged into a HI Coater perforated pan coater and preheated to 30° C. to 32° C. at a tumbling rate of 15 rpm. The tablets were coated using the following parameters:

| Parameter | Value |
|---|---|
| Inlet air temperature | 40° C. |
| Outlet air temperature | 30° C. |
| Air flow | 70 m³/hr |
| Pan rotation | 10 rpm |
| Air pressure in die | 1.0 bar |

The coating was continued until the tablets had gained on the average 13 mg coating.

In Vitro Drug Release Tests

The tablets were tested for two hours for drug release in 900 ml 0.1N HCl at 37° C. and 50 rpm using a USP type II dissolution bath. Drug content was determined by HPLC using the following conditions:

Column: 250×4.6 mm, 5μ Hypersil BDS C18.

Mobile phase: 2.5% acetonitrile and 97.5% 0.1N phosphate buffer pH=2.8.

Flow rate and retention time: 1.2 ml/min, 4.0 min.

Detector parameters: UV at 280 nm.

The tablets showed 0% drug release in the acidic medium.

The tablets were tested for six more hours for drug release in 900 ml 0.1 N phosphate buffer of pH=6.8 at 37° C. and 50 rpm using a USP type II dissolution bath and analyzed by HPLC using the same conditions as above. The results of the drug release were as follows:

| Time (hr) | Average % cumulative release | % RSD |
|---|---|---|
| 3 | 40.6 | 13.9 |
| 4 | 69.4 | 13.75 |
| 5 | 85.7 | 10.4 |
| 6 | 95.4 | 6.5 |
| 7 | 99.7 | 2.9 |
| 8 | 101.6 | 1.5 |

Outer Mantle—Levodopa/Carbidopa

Carbidopa/levodopa granulate: Carbidopa (90 grams), levodopa (180 g), and polyvinylpyrrolidone (Povidone K-30™, 30 grams) were added to a Diosna P1/6 high shear granulator and mixed for 5 minutes at 380 rpm. Over the next minute ethanol (95%, 60 ml) was added as a granulating solvent while the mass was being mixed at 380 rpm. The mixture was then massed at 380 rpm for 2 minutes. The wet granulate was milled through a 2.5 mm screen in an Erweka mill and subsequently dried for 35 minutes in an Aeromatic Lab fluidized bed drier to 2.8% volatiles at an inlet temperature of 30° C. The dry granulate was milled once again through a 0.8 mm screen.

Tableting mixture: The milled, dry, carbidopa/levodopa granulate (250 grams) was placed in a 5 liter V mixer. Cellactose 80™(271 grams) and crospovidone (43 grams) were added and mixed in the V mixer for 5 minutes. Magnesium stearate NF/EP (6 grams) was added and the V mixer operated for a further half a minute.

Tablet formation: The enteric coated levodopa inner cores containing 100 mg levodopa were pressed with the tabletting mixture using the special spring loaded core rod tooling for making annular sheathed tablets. The lower punch was an oval flat beveled punch of 17.6 mm by 10 mm with an inner hole (for the core rod) of 10.6 mm by 6.35 mm oblong shape. The upper punch was a flat beveled punch of 17.6 mm by 10 mm with an oblong protrusion that was 1.0 mm tall and 10.6 mm by 6.35 mm oblong shape with slight tapering. Each tablet formed contained 100 mg levodopa and 50 mg carbidopa in the outer annular sheath and 100 mg levodopa in the enteric coated core.

### Example 4

### Pharmacokinetic Trial of the Dosage Form of Example 3

A single dose pharmacokinetic trial was carried out with 12 healthy volunteers on the tablets from Example 3 as test tablets and Sinemet-CR® (Levodopa/Carbidopa 200/50 mg—Merck & Co. Inc.) as the reference tablets. Blood samples were taken at 0 time (before dosing) and 0.5, 1, 2, 3, 4, 5, 6, 7, 8, 9, 10, 11, 12, 16 and 24 hours post dosing. Plasma concentrations and pharmacokinetic parameters were determined as in Example 2.

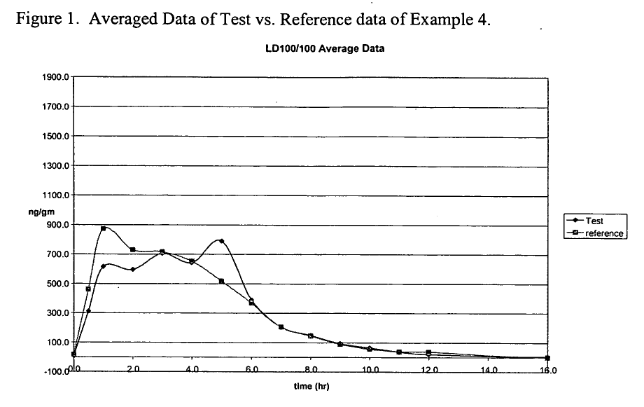

Results of the raw data obtained for the test and reference drugs are presented in Table 6A and Table 6B respectively while the pharmacokinetic parameters that were calculated therefrom are presented in Table 7. A graph of the averaged data over the patient population is given in FIG. 1.

TABLE 6A
Levodopa Concentrations in ng/ml using Administration 1(A): 1 × 200 mg Dual-Release Protected
Tablet Levodopa/50 mg Carbidopa TEST TABLET 1 for a total unit dose of 200 mg LD and 50 mg CD.

| | Subject Number | | | | | | | | | | | |
|---|---|---|---|---|---|---|---|---|---|---|---|---|
| Time | 01 | 02 | 03 | 04 | 05 | 06 | 07 | 08 | 09 | 10 | 11 | 12 |
| 1:00:00 | BLQ | BLQ | BLQ | BLQ | BLQ | BLQ | BLQ | BLQ | 82.13 | BLQ | BLQ | BLQ |
| 1:00:30 | 666.92 | BLQ | 110.03 | 173.86 | 368.79 | 625.41 | 162.11 | 220.16 | 282.52 | 321.29 | 326.47 | 480.42 |
| 1:01:00 | 412.57 | 94.64 | 1021.74 | 533.46 | 571.08 | 596.30 | 258.29 | 1238.33 | 668.43 | 1040.81 | 261.91 | 688.94 |
| 1:02:00 | 832.38 | 635.73 | 799.57 | 343.26 | 285.54 | 691.11 | 692.77 | 488.93 | 608.22 | 560.52 | 556.90 | 645.40 |
| 1:03:00 | 359.76 | 704.70 | 567.89 | 889.33 | 1440.92 | 1552.44 | 297.96 | 368.83 | 393.76 | 618.87 | 474.81 | 794.65 |
| 1:04:00 | 397.85 | 419.09 | 1124.57 | 450.98 | 957.78 | 657.94 | 257.33 | 471.24 | 877.55 | 726.13 | 271.87 | 1093.88 |
| 1:05:00 | 370.43 | 1522.19 | 543.92 | 1873.09 | 434.63 | 312.81 | 1241.31 | 1159.39 | 485.74 | 366.78 | 625.91 | 537.01 |
| 1:06:00 | 204.24 | 572.66 | 348.66 | 849.36 | 242.02 | 223.24 | 465.45 | 412.09 | 277.57 | 184.12 | 623.35 | 276.76 |
| 1:07:00 | 107.90 | 208.76 | 186.09 | 386.19 | 200.50 | 157.43 | 262.73 | 313.82 | 159.50 | 99.57 | 227.35 | 162.95 |
| 1:08:00 | 77.53 | 143.44 | 176.24 | 247.62 | 134.44 | 116.48 | 167.17 | 276.55 | 108.48 | 80.38 | 130.92 | 113.33 |
| 1:09:00 | 40.15 | 69.99 | 89.50 | 153.67 | 96.35 | 69.58 | 108.85 | 127.58 | 101.18 | 57.52 | 113.59 | 90.65 |
| 1:10:00 | BLQ | 51.15 | 113.52 | 113.81 | 110.46 | 56.20 | 58.06 | 103.13 | 69.17 | 48.71 | 39.48 | BLQ |
| 1:11:00 | BLQ | 32.66 | BLQ | 88.33 | 75.92 | 39.21 | 45.91 | 78.13 | 43.18 | BLQ | 37.48 | BLQ |
| 1:12:00 | BLQ | 511.81 | BLQ | 87.88 | BLQ | BLQ | BLQ | 110.87 | BLQ | BLQ | 40.51 | BLQ |
| 1:16:00 | BLQ | BLQ | BLQ | BLQ | BLQ | BLQ | BLQ | BLQ | BLQ | 41.23 | BLQ | BLQ |
| 2:00:00 | BLQ | 88.17 | BLQ | BLQ | BLQ | BLQ | BLQ | BLQ | BLQ | BLQ | BLQ | BLQ |

BLQ: Below the lower limit of quantitation (30.49 ng/ml)

TABLE 6B
Levodopa Concentrations in ng/ml using Administration 3(C): 1 × Sinemet-CR tablet (Levodopa/
Carbidopa 200/50 mg; Merck & Co., Inc.), for a total unit dose of 200 mg LD and 50 mg CD; Reference

| | Subject Number | | | | | | | | | | | |
|---|---|---|---|---|---|---|---|---|---|---|---|---|
| Time | 01 | 02 | 03 | 04 | 05 | 06 | 07 | 08 | 09 | 10 | 11 | 12 |
| 1:00:00 | BLQ | BLQ | BLQ | 104.68 | BLQ | BLQ | BLQ | 82.31 | BLQ | BLQ | BLQ | BLQ |
| 1:00:30 | 781.99 | 61.41 | 33.33 | 334.22 | 1067.13 | 1134.72 | 88.70 | 165.73 | 868.99 | 81.61 | 381.64 | 530.99 |
| 1:01:00 | 1430.63 | 528.95 | 292.21 | 319.76 | 688.30 | 1269.45 | 659.81 | 1192.82 | 1086.05 | 1420.01 | 553.72 | 1023.13 |
| 1:02:00 | 650.91 | 651.35 | 593.45 | 405.91 | 412.17 | 1076.53 | 781.35 | 613.02 | 860.67 | 967.95 | 890.11 | 843.09 |
| 1:03:00 | 725.39 | 523.85 | 1237.41 | 423.08 | 497.68 | 851.35 | 506.80 | 467.29 | 900.12 | 682.48 | 992.94 | 773.00 |
| 1:04:00 | 691.54 | 491.16 | 846.91 | 1207.09 | 404.90 | 838.37 | 526.53 | 386.01 | 544.39 | 585.20 | 640.09 | 666.96 |
| 1:05:00 | 611.13 | 948.40 | 1013.22 | 764.27 | 163.99 | 404.60 | 205.17 | 530.89 | 397.82 | 279.98 | 365.83 | 524.09 |
| 1:06:00 | 342.93 | 657.40 | 581.99 | 486.18 | 111.39 | 399.90 | 388.76 | 469.43 | 253.97 | 146.25 | 259.13 | 328.04 |
| 1:07:00 | 239.47 | 438.64 | 353.88 | 212.59 | 78.29 | 166.69 | 110.72 | 272.69 | 157.64 | 97.73 | 161.39 | 186.52 |
| 1:08:00 | 218.07 | 192.13 | 233.08 | 170.80 | 75.45 | 187.50 | 43.25 | 199.19 | 126.86 | 66.68 | 92.61 | 139.88 |
| 1:09:00 | 108.42 | 91.89 | 150.00 | 114.00 | 67.99 | 86.18 | 39.10 | 124.85 | 61.77 | 36.98 | 56.25 | 123.57 |
| 1:10:00 | 56.07 | 52.30 | 93.39 | 102.63 | BLQ | 57.67 | BLQ | 88.70 | 99.85 | 44.29 | 32.18 | 48.35 |
| 1:11:00 | 50.47 | BLQ | 58.36 | BLQ | BLQ | 57.08 | 151.49 | 104.68 | 33.71 | BLQ | BLQ | BLQ |
| 1:12:00 | 32.77 | BLQ | 64.92 | 35.11 | BLQ | 32.09 | 167.21 | 111.32 | BLQ | BLQ | BLQ | BLQ |
| 1:16:00 | BLQ | BLQ | BLQ | BLQ | BLQ | BLQ | BLQ | BLQ | BLQ | BLQ | BLQ | BLQ |
| 2:00:00 | BLQ | BLQ | BLQ | BLQ | BLQ | BLQ | BLQ | BLQ | BLQ | BLQ | BLQ | BLQ |

BLQ: Below the lower limit of quantitation (30.49 ng/ml)

TABLE 7
Pharmacokinetic Parameters Test Tablet vs. Reference

| | AUC | AUCinf | | | | Tmax | Cmax | Cmaxtest/ | AUCtest/ |
|---|---|---|---|---|---|---|---|---|---|
| vol-sess | (h*ng/g) | (h*ng/g) | t_{1/2} | CL | Vd | (h) | (ng/g) | Cmaxref | AUCref |
| 1 (test) | 3033.1 | 3033.1 | 1.3 | 65.9 | 126.5 | 2.0 | 832.4 | 0.58 | 0.58 |
| 2 (test) | 4784.0 | 4872.2 | 1.5 | 41.8 | 88.2 | 5.0 | 1522.2 | 1.61 | 1.07 |
| 3 (test) | 4771.3 | 4771.3 | 1.7 | 41.9 | 101.4 | 4.0 | 1124.6 | 0.91 | 0.86 |
| 4 (test) | 6102.4 | 6102.4 | 2.6 | 32.8 | 124.8 | 5.0 | 1873.1 | 1.55 | 1.38 |
| 5 (Test) | 4591.3 | 4591.3 | 2.2 | 43.6 | 137.7 | 3.0 | 1440.9 | 1.35 | 1.60 |
| 6 (test) | 4636.4 | 4636.4 | 2.0 | 43.1 | 122.7 | 3.0 | 1552.4 | 1.22 | 0.81 |
| 7 (test) | 3872.3 | 3872.3 | 1.4 | 51.6 | 104.2 | 5.0 | 1241.3 | 1.59 | 1.04 |
| 8 (test) | 5115.7 | 5115.7 | 1.7 | 39.1 | 97.6 | 1.0 | 1238.3 | 1.04 | 1.13 |
| 9 (test) | 3787.5 | 3787.5 | 2.0 | 52.8 | 155.6 | 4.0 | 877.6 | 0.81 | 0.81 |
| 10 (test) | 3931.2 | 3931.2 | 1.9 | 50.9 | 137.3 | 1.0 | 1040.8 | 0.73 | 0.98 |
| 11 (test) | 3562.6 | 3562.6 | 1.4 | 56.1 | 116.9 | 5.0 | 625.9 | 0.63 | 0.87 |
| 12 (test) | 4471.5 | 4471.5 | 1.9 | 44.7 | 120.5 | 4.0 | 1093.9 | 1.07 | 0.96 |
| 1 (ref) | 5240.3 | 5240.3 | 1.7 | 38.2 | 92.8 | 1.0 | 1430.6 | | |
| 2 (ref) | 4474.5 | 4474.5 | 1.0 | 44.7 | 67.5 | 5.0 | 948.4 | | |
| 3 (ref) | 5559.8 | 5559.8 | 1.7 | 36.0 | 90.7 | 3.0 | 1237.4 | | |
| 4 (ref) | 4407.4 | 4407.4 | 1.7 | 45.4 | 114.6 | 4.0 | 1207.1 | | |
| 5 (ref) | 2861.7 | 2861.7 | 4.6 | 69.9 | 460.4 | 0.5 | 1067.1 | | |
| 6 (ref) | 5725.5 | 5725.5 | 1.8 | 34.9 | 89.8 | 1.0 | 1269.5 | | |
| 7 (ref) | 3710.4 | 3710.4 | 0.9 | 53.9 | 68.8 | 2.0 | 781.4 | | |
| 8 (ref) | 4533.1 | 4533.1 | 1.7 | 44.1 | 107.2 | 1.0 | 1192.8 | | |
| 9 (ref) | 4685.8 | 4685.8 | 2.0 | 42.7 | 122.6 | 1.0 | 1086.1 | | |
| 10 (ref) | 4013.4 | 4013.4 | 2.1 | 49.8 | 148.3 | 1.0 | 1420.0 | | |
| 11 (ref) | 4096.6 | 4096.6 | 1.3 | 48.8 | 93.4 | 3.0 | 992.9 | | |
| 12 (ref) | 4666.3 | 4666.3 | 1.6 | 42.9 | 101.1 | 1.0 | 1023.1 | | |
| AVG(test) | 4388.3 | 4395.6 | 1.8 | 47.0 | 119.5 | 3.5 | 1205.3 | 1.091 | 1.008 |
| AVG (ref) | 4497.9 | 4497.9 | 1.8 | 45.9 | 129.8 | 2.0 | 1138.0 | | |
| geomn(test) | 4320.1 | 4326.7 | 1.8 | 46.3 | 118.0 | 3.1 | 1156.7 | 1.030 | 0.975 |
| geomn(ref) | 4428.7 | 4428.7 | 1.7 | 45.2 | 110.8 | 1.5 | 1122.7 | | |
| stddev(test) | 811 | 815 | 0.38 | 8.85 | 19.24 | 1.51 | 350.71 | 0.37 | 0.27 |
| stddev(ref) | 795 | 795 | 0.93 | 9.37 | 106.47 | 1.45 | 191.93 | | |
| % CV (test) | 18.48% | 18.55% | 21.17% | 18.82% | 16.10% | 43.07% | 29.10% | | |
| % CV (ref) | 17.68% | 17.68% | 50.11% | 20.41% | 82.04% | 74.21% | 16.87% | | |

From the pharmacokinetic data calculated and presented in Table 7 one can see that the AUC (area under the curves) and the Cmax values are similar for the two administrations whether averaged over the populations or averaged after determining the ratio for each individual as his own control with the geometric means being within 3% of each other for both values. From this data, one can conclude that the bioavailability of the levodopa combination is not impaired by half the dose (100 mg) being delayed until after leaving the stomach and the duodenum. The Test formulation had an average Tmax (3.5 hr vs. 2.0 hr) that was prolonged compared to the reference which was in itself a controlled release (but not a delayed release) formulation. The graph in FIG. 1 of the averaged data showed an improved profile for the test formulation with the levodopa concentrations being more of a plateau and peaking at a later time than the reference formulation.

What is claimed is:

1. A pharmaceutical dosage form comprising a first dose of about 30 to 500 mg of levodopa, wherein the levodopa is released after passage through the stomach and duodenum.

2. The dosage form of claim 1, wherein the levodopa is released at least 10 minutes after passage through the stomach.

3. The dosage form of claim 1 further comprising at least one carboxylase enzyme inhibitor.

4. The dosage form of claim 3, wherein the carboxylase enzyme inhibitor is carbidopa or benseazide.

5. The dosage form of claim 1, wherein the levodopa is formulated as an immediate release formulation.

6. The dosage form of claim 1, wherein the levodopa is released during about 2 to 5 hours.

7. The dosage form of claim 1 further comprising a second dose of levodopa, wherein the second dose begins to release levodopa upon ingestion into the stomach.

8. The dosage form of claim 7, wherein the second dose of levodopa is formulated as an immediate release formulation.

9. The dosage form of claim 7, wherein the second dose of levodopa is released over about 2 to 5 hours.

10. The dosage form of claim 1, wherein the dosage form is an enterically coated tablet, an enterically coated capsule, or enterically coated pellets.

11. The dosage form of claim 10, wherein the dosage form is an enterically coated tablet.

12. The dosage form of claim 1, wherein the dosage form is an erosive delay coated tablet, erosive delay coated capsule or erosive delay coated pellet.

13. The dosage form of claim 7, wherein the first dose is an enterically coated tablet and the second dose is an annular sheath.

14. The dosage form of claim 7, wherein the first and second dose each are a layer of a bilayer tablet.

15. The dosage form of claim 7, wherein the first and second dose are two populations of pellets in a capsule.

16. The dosage form of claim 7, wherein the first dose is enterically coated pellets embedded in a matrix.

17. A method of treating Parkinson's disease comprising administering to a patient in need of such treatment a first dose of about 30 mg to 500 mg of levodopa, wherein the first dose releases levodopa after passage though the stomach and the duodenum

18. The method of claim 17, wherein the levodopa releases at least 10 minutes after leaving the stomach.

19. The method of claim 17 further comprising dosing at least one carboxylase enzyme inhibitor.

20. The method of claim 19, wherein the carboxylase enzyme inhibitor is carbidopa or benseazide.

21. The method of claim 17, wherein the first dose of levodopa is formulated as an immediate release formulation.

22. The method of claim 17, wherein the levodopa is released during 2 to 5 hours.

23. The method claim 17 further comprising a second dose of levodopa, wherein the second dose begins to release immediately upon ingestion into the stomach.

24. The method of claim 23, wherein the second dose of levodopa is formulated as an immediate release formulation.

25. The method of claim 23, wherein the second dose of levodopa is released over 2 to 5 hours.

26. The method of claim 17, wherein the first dose is an enterically coated tablet, an enterically coated capsule, or enterically coated pellets.

27. The method of claim 26, wherein the first dose is an enterically coated tablet.

28. The method of claim 17, wherein the first dose is an erosive delay coated tablet, erosive delay coated capsule, or erosive delay coated pellet.

29. The method of claim 23, wherein the first dose is an enterically coated tablet and the second dose is an annular sheath.

30. The method of claim 23, wherein the first and second doses each are a layer of a bilayer tablet.

31. The method of claim 23, wherein the first and second doses each a population of pellets in a capsule.

32. The method of claim 17, wherein the first dose is an enterically coated pellets embedded in a matrix.

33. The method of claim 23, wherein the first and second dose are separate dosage forms.
